(19) United States
(12) Patent Application Publication
(10) Pub. No.: US 2022/0016625 A1
(43) Pub. Date: Jan. 20, 2022

(54) FLOW CELL USING PELTIER MODULE AS PRIME MOVER FOR POLYMERASE CHAIN REACTION

(71) Applicant: Siemens Healthcare Diagnostics Inc., Tarrytown, NY (US)

(72) Inventor: Daniel Chu, Hercules, CA (US)

(73) Assignee: Siemens Healthcare Diagnostics Inc., Tarrytown, NY (US)

(21) Appl. No.: 17/309,319

(22) PCT Filed: Nov. 15, 2019
(86) PCT No.: PCT/US2019/061607

§ 371 Date: May 19, 2021

Related U.S. Application Data
(60) Provisional application No. 62/793,628, filed on Jan. 17, 2019.

(51) Int. Cl. B01L 3/00; B01L 7/00

(52) U.S. Cl. CPC B01L 3/502715; C12Q 1/686; B01L 3/502746; B01L 7/52

(57) ABSTRACT

A flow cell for oscillating flow PCR has pumping action via thermally-induced internal pressure variations. Rapid movement of a sample comprised of target DNA and associated reagents between heated zones within the flow cell for oscillating flow PCR is achieved without mechanical moving parts and without contamination. A channel extends from a loading port to first and second heated zones and to a central air chamber. The sample is movable between the heated zones in response to central air chamber pressure changes induced by external thermal changes. The flow cell is insertable into a flow cell process heater for heating each heated zone to a respective temperature. The central air chamber is aligned above a flow control heater for thermally inducing the internal pressure changes in the channel.

### FIELD OF THE DISCLOSURE

The disclosure herein relates generally to the field of oscillating flow cells for use in amplification of DNA sequences, and more particularly to a system and method for moving a mix of template DNA and associated reagents within a flow cell for Polymerase Chain Reaction (PCR), Quantitative PCR (qPCR) and other, similar techniques without mechanical means such as pumps or valves.

### BACKGROUND

PCR and qPCR are well-known techniques for the amplification of target DNA sequences. A mix typically comprising at least the target DNA sample, primers, nucleotides, and DNA polymerase is subject to multiple temperature cycles, including a denaturation step carried out at about 94-98° C., an annealing step carried out at about 50-65° C., and an extension step. The latter may be carried out at around 70° C. but, depending upon the DNA polymerase used, may also be carried out at the same temperature as the annealing step.

In qPCR, the PCR product may be detected in real time during the amplification process as a result of fluorescent reporters. Fluorescent nucleotide sequence probes have a fluorescent report at one end and a fluorescence quencher at the opposite end. The probes, like primers, anneal to single-stranded DNA during the annealing phase of PCR. They are then degraded by DNA polymerase during the elongation phase. The released reporter fluorophore is thus detectable.

A microfluid chip or flow cell is a set of micro-channels etched or molded into a material such as glass, silicon, or polymer (e.g., PolyDiMethylSiloxane (PDMS)). The micro-channels forming the microfluidic chip are connected together in order to achieve the desired features. External actuation means are used to direct the transport of the media within the micro-channels. For example, external drives may be used to impart centrifugal forces on passive chips.

Active components may also be integrated with or within a microfluidic chip or flow cell to control the media flow. Micropumps supply fluids in a continuous manner, while microvalves control the flow direction and/or selective movement of pumped fluids. However, external drives require controllers for selective operation as well as physical space to physically manipulate the passive chips.

Micropumps and valves may be integrated into the micro-channel itself. Certain flow cells have been developed with a Peltier pump and associated valve for selective fluid movement. However, the design and manufacture of a valve with the required flow rate response head is very difficult. Systems with integrated active components such as micropumps and microvalves require micro-pneumatic systems for controlling the selective movement of fluid within the micro-channels. Rapid and repeatable precision of fluid movement within a microfluidic chip is imperative for applications such as PCR and qPCR.

Rather than moving fluid within a microfluidic chip or flow cell between different temperature zones, target fluid flows may be selectively heated to a desired temperature through use of a variable temperature element. However, precise control over the fluid temperature is complicated and may result in slower temperature response in the target fluid.

Alternatively, PCR samples may be rapidly transferred between different heating blocks maintained at desired PCR temperatures in a continuous flow or flow-through PCR cell. A pump is required to move the PCR mix through a microfluidic channel that typically follows a serpentine path between temperature zones. Drawbacks associated with this option include having a fixed number of temperature cycles and the need for an external pump.

Another approach to thermal cycling in the microfluidic context is oscillating flow PCR. By enabling the selective adjustment of the direction of fluid flow within the flow cell, the number of temperature cycles is selectable, thus solving a drawback associated with continuous-flow PCR. However, means must be provided for the selective adjustment or routing of the fluid flows. One approach to solving this problem utilizes a Quake valve to circulate PCR mix between different temperature zones. However, a precision-controllable air pump is thus required for implementing the valving action.

What is absent in the prior art though highly desirable is a microfluid chip or flow cell for use in DNA amplification processes such as PCR or qPCR that is capable of providing repeated and accurate movements of fluid, without mechanical moving parts, pumps, or valves, while preventing contamination.

### SUMMARY

In order to overcome the inability of the prior art to provide the functionality of a flow cell for oscillating flow PCR without the use of pumps or other mechanical parts, the present disclosure provides a low cost, sealed flow cell for oscillating flow PCR that has pumping action via thermally induced internal pressure variations.

The foregoing system enables rapid movement of target DNA and associated reagents between heated zones within a flow cell for oscillating flow PCR without mechanical moving parts and without contamination.

The flow cell includes a microchannel that extends from a loading port to first and second heated zones and to a central air chamber that is selectively heatable and coolable by a Peltier module disposed therebeneath. A fourth ambient temperature zone is also in communication with the channel and represents a reference zone for the pneumatic system of the channel. A sample comprised of target DNA and associated reagents is movable from the second heated zone to the first heated zone by an increase in central air chamber pressure resulting from heat applied by the underlying Peltier module. The sample is also movable from the first heated zone to the second heated zone by a decrease in central air chamber pressure resulting from a reduction in heat, or by cooling, applied by the underlying Peltier module.

The flow cell is insertable into a flow cell process heater having at least two heaters for heating the first and second zones of the flow cell to a respective, desired temperature. When used for DNA amplification in processes such as PCR or qPCR, the first zone is heated to a desired denaturation temperature while the second zone is heated to a desired annealing temperature.

Once inserted into the flow cell process heater, the central air chamber is aligned above a flow control heater having the underlying Peltier module. The thermal expansion and contraction of air or other gas inside the central air chamber, resulting from heating or cooling applied by the underlying Peltier module, pushes or pulls the sample back and forth between the first and second heated zones.

Very fast PCR results can be achieved and the flow cell can have a long service life as the Peltier module does not require high temperature cycles for moving the sample between the heated zones. Simplicity in manufacturability and cost savings are achieved through the ability to rapidly and accurately move PCR sample within the flow cell without the use of mechanical moving parts and without contamination.

### BRIEF DESCRIPTION OF THE DRAWINGS

Illustrative embodiments of the disclosed technology are described in detail below with reference to the attached drawing figures, which are incorporated by reference herein and wherein:

FIG. 1 is a perspective view of a flow cell for use with a flow cell process heater and a flow control heater according to the present invention;

FIG. 2 is a partial perspective view of a flow cell process heater and a flow control heater for use with the flow cell of FIG. 1; and

FIG. 3 is a partial perspective view of the flow cell of FIG. 1 received within the flow cell process heater and the flow control heater of FIG. 2.

### DETAILED DESCRIPTION

Disclosed herein is a flow cell, flow cell process heater, and flow control heater that collectively enable the selective movement of a DNA template and associated reagents within the flow cell for processes such as DNA amplification without the use of mechanical means such as pumps and/or valves. The DNA template and associated reagents are alternatively and collectively referred to herein simply as “sample.”

While the context of the present disclosure is in the field of DNA amplification via techniques such as PCR and qPCR, it is understood that the presently disclosed components and techniques also find utility in other microfluidic applications.

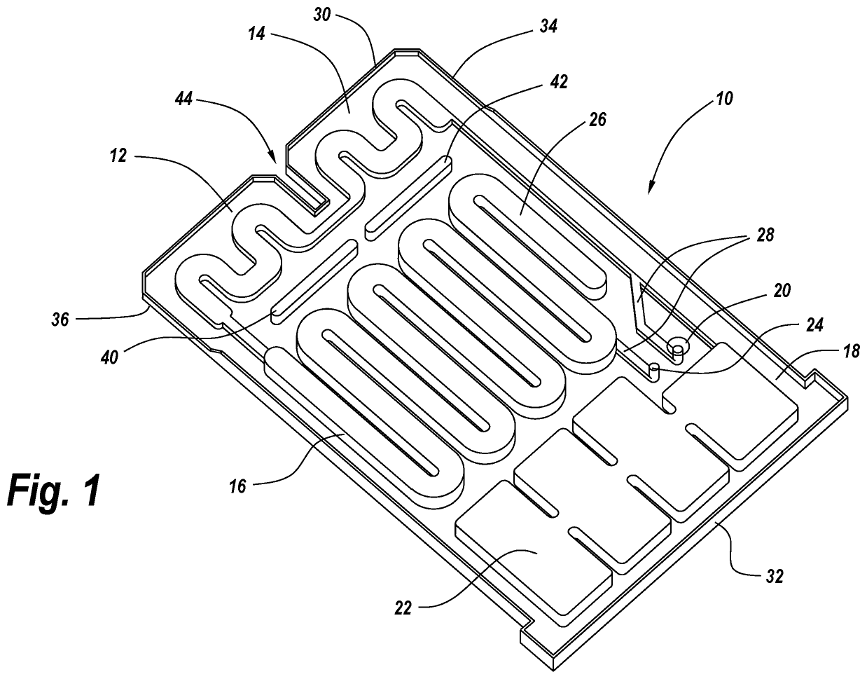

With regard to FIG. 1, the flow cell 10 is shown as a rectangular chip having a distal end 30, a proximal end 32, a first side 34, and a second side 36. An upper face of the flow cell is generally visible in FIG. 1, while an opposite lower face is not shown. The lower surface in a first embodiment is featureless and planar and may be provided with a layer of material of high thermal conductivity such as aluminum.

The upper face comprises four principal regions. The first region is an annealing region 14, also referred to as the first heat portion, proximate the distal end and first side. The second region is a denaturation region 12, also referred to as the second heat portion, proximate the distal end and second side. The third region is a middle, transport region 16, also referred to as the third heat portion, intermediate the distal and proximal ends and between the first and second sides. The fourth region is an ambient reference region 18, also referred to as the fourth unheated portion, proximate the proximal end.

A fluid flow path or channel 26 is connected between each of the four regions, thereby forming a continuous fluid flow channel therebetween. The channel may be formed through a variety of techniques, including providing a lower base layer and bonding an upper layer, having the microchannel etched or otherwise formed therein, onto the base layer, being careful to completely bond the two layers to avoid air leaking into the channel via the layer interface. One or both layers may be formed of PDMS, polypropylene, polycarbonate, or any other suitable material, depending upon the application, such as PCR or qPCR.

The fluid flow path or channel 26 is preferably provided as a looped or serpentine path in the first and second regions 12, 14 for enhanced thermal transfer to the fluid, discussed subsequently. A serpentine path also lengthens the path, thereby providing an additional or “overflow” region to compensate for variability in the sample volume and loading fluid injection. The fluid flow path or channel 26 in the third portion 16 is preferably larger in volume to also facilitate thermal transfer to gas within the local region of the fluid flow path or channel, as well as to provide a relatively large volume of gas the pressure of which is manipulated by heat from the flow control heater 200, also discussed subsequently. The fluid flow path or channel 26 in the fourth region 18 is preferably provided with a large surface area, without the need for a serpentine path, as this region serves as a pressure reference point for the remainder of the fluid flow path or channel.

Once the flow cell 10 has been installed with respect to the flow cell process heater and flow control heater, as described in greater detail below, a relatively small quantity of DNA template and associated reagents, for example about 5 μL, is introduced into the flow path via a loading port 20 which may be within the ambient reference region 18. DNA template and reagent introduction may be via a manual or automatically manipulated pipette (not shown).

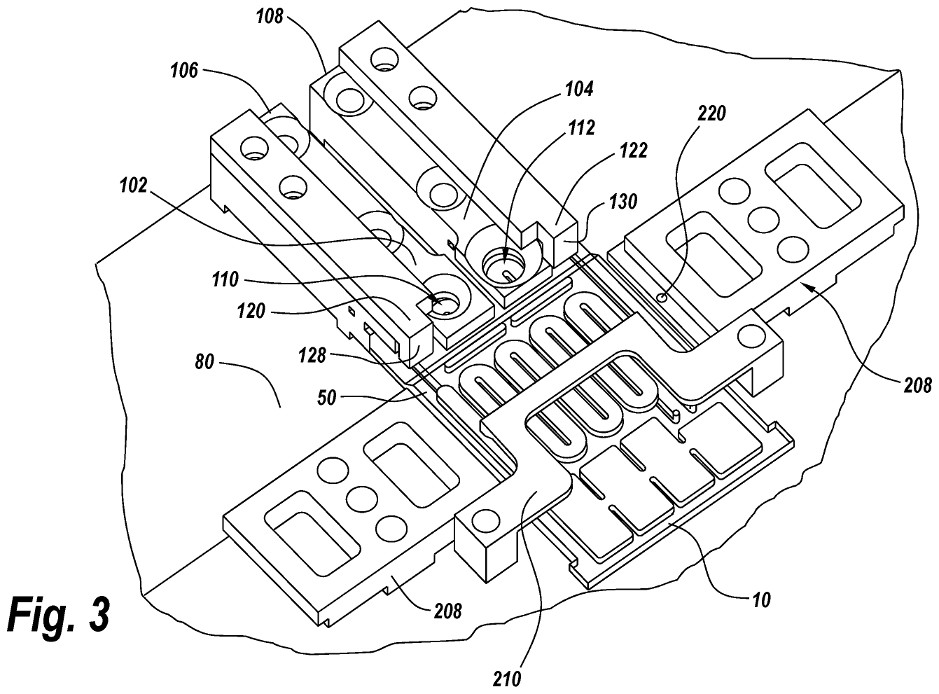

Visible in FIG. 3 is a lid 50 disposed over the third portion 16 and fourth portion 18. The loading port 20 and a vent port 24 (discussed below) are formed through the lid. In one embodiment, the fluid flow channel 26 is formed in the bottom of the lid and the lid forms the top of the flow cell 10.

Because of the orientation of the fluid channel 26, the DNA template and associated reagents, once introduced via the sample loading port 20, flow towards the denaturation region 12 and annealing region 14. A volume of inert fluid such as air and/or mineral oil is introduced through the sample loading port to urge the sample to a point intermediate the denaturation region 12 and annealing region 14. The use of mineral oil is beneficial in preventing contamination of the DNA template and reagents. The volume of inert fluid introduced following the DNA template and reagents is selected to advance the DNA templates and reagents to a desired location with respect to the denaturation region 12 and annealing region 14.

A vent port 24 is connected to a distal end of the fluid channel 26 to enable the DNA template and reagents, and following inert fluid, to be injected into the fluid channel without the resistance of increased pressure associated with a closed channel. The vent port may be within the ambient reference region 18. Once so injected and located at the desired position relative to the denaturation region 12 and annealing region 14, the sample loading port 20 and vent port may be cut off from the fluid channel to form a closed flow cell system such as through deformations 28 mechanically formed proximate each in the fluid channel. Such deformations may be the result of an externally applied force that collapses or otherwise creates a discontinuity in the fluid channel at each point. The injection of a volume of mineral oil into the loading port, or depositing a volume of mineral oil onto the upper face of the flow cell 10 proximate the loading port, may obviate the need for physically closing off the loading port.

In certain embodiments, such as shown in FIG. 1, thermal barriers may be employed to avoid the migration of thermal effects from one region to another, adjacent region. For example, a closed channel or other physical barrier 40 may be disposed on the upper face of the flow cell 10 between the second region 12 and the third region 16. Similarly, a barrier 42 may be disposed between the first region 14 and the third region. While a similar structure may also be disposed intermediate the first and second regions, there may be several benefits to the use of a physical discontinuity such as a notch 44, as shown in FIG. 1. Specifically, while providing the desired thermal barrier, such a notch may also facilitate proper alignment and installation of the flow cell into a flow cell process heater 100 if the flow cell process heater is provided with a complimentary alignment feature.

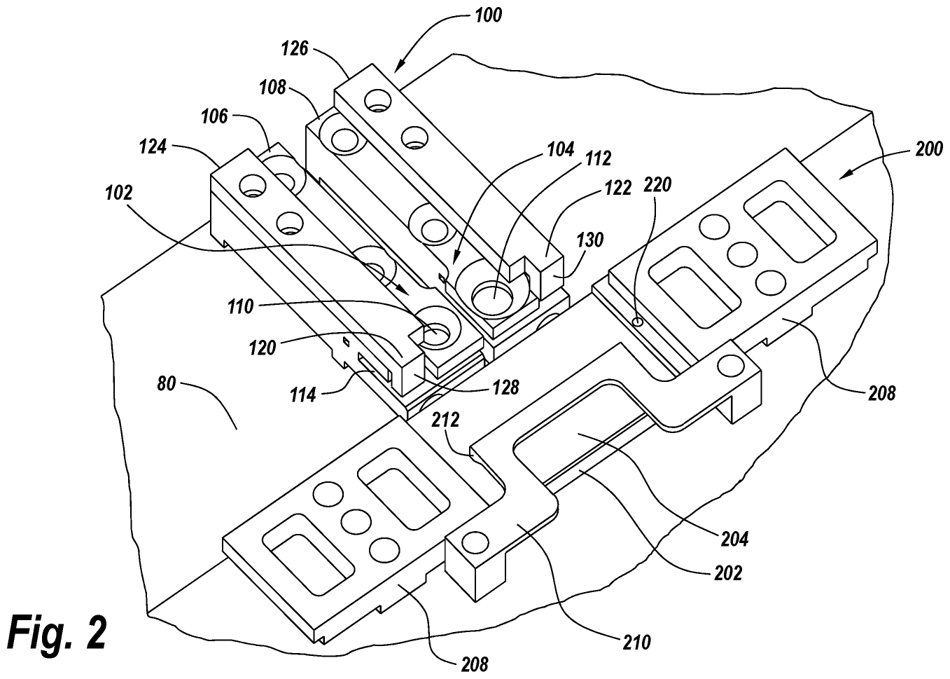

With respect to FIG. 2, a flow cell process heater 100 and a flow control heater 200 are shown, without the flow cell 10. In a first embodiment, both elements are disposed with respect to a substrate 80 such as a heat sink.

The flow cell process heater 100, in the embodiment shown in FIG. 2, comprises two portions 102, 104, each of which may be attached to the heat sink 80 via reversible fasteners such as screws (not shown). The first portion 102 comprises a first flow cell process heater 106, while the second portion comprises a second flow cell process heater 108. Each of the first and second flow cell process heaters may be selected as a simple resistive-type heater capable of maintaining a predefined or predetermined temperature, or may be provided as a variable-temperature heater such as a ThermoElectric Device (TED), for example, a Peltier device. Non-variable temperature devices may be useful in environments where melt curve for target DNA and reagents are already established, whereas variable temperature devices may facilitate PCR melt curve studies.

One end of the flow cell process heater 100 has a lateral notch 114 configured for selectively receiving the distal end 30 of the flow cell 10. Once the flow cell is received within the flow cell process heater, as discussed with respect to FIG. 3, detection windows 110, 112 enable optical detection devices (not shown) to monitor the activity within the flow cell, including measuring fluorescence in the case of qPCR. Optical detection may be via photodiode or a camera-based detector.

Above each of the heaters 106, 108 in each portion 102, 104 of the flow cell process heater 100, a respective resilient member 120, 122 extends laterally across the top of the heater. A distal end 124, 126 of each is fixedly attached to a top surface of the respective heater, while a downwardly projecting proximal end 128, 130 is free to deflect upwards, as will be discussed subsequently. In a first embodiment, the resilient members are not heat conducting and may be formed of heat-resistant plastic, for instance.

The flow control heater 200 comprises a heater module 202, which in a first embodiment is a variable temperature Peltier module. Disposed above the heater module in an exemplary embodiment is a heat distribution plate 204 selected for good thermal conductivity. For example, the heat distribution plate may be provided of aluminum. Alternatively, other heat conductive materials may be employed, such as steel or brass. The heater module may be maintained in place with respect to the substrate 80 by a pair of clamps 208 that may be attached to the substrate via reversible fasteners such as screws (not shown).

The flow control heater 200 also comprises a resilient flow cell retention clip 210 attached to the substrate 80, such as through the use of reversible fasteners such as screws (not shown). The retention clip preferably extends laterally above the heat distribution plate 204 and heater module 202 therebeneath. A laterally extending downward projection 212 may be provided on the retention clip for the purpose of mechanically interfering with a flow cell 10 once installed within the flow cell process heater 100 and the flow control heater, as discussed subsequently. In a first embodiment, the retention clip is formed of a heat conductive material, such as aluminum, brass, or steel, thereby ensuring efficient thermal coupling between the flow control heater and the third portion 16 of the flow cell 10.

Lastly, the flow control heater 200 may comprise a temperature sensor 220 disposed in conjunction with the heat distribution plate 204 for monitoring the performance of the heater module 202. An output of the temperature sensor may be provided to a control circuit (not shown) also associated with the heater module 202 for providing a feedback loop thereto.

With respect to FIG. 3, the flow cell 10 has been inserted into the notch 114 (FIG. 2) in the end of the flow cell process heater. Thus, a portion of the fluid flow channel 26 in the second region 12 is visible in one detection window 110 while a portion of the fluid flow channel in the first region 14 is visible in the other detection window 112. The downwardly projecting proximal ends 128, 130 of the resilient members 120, 122 come into contact with the upper face of the flow cell upon insertion and are deflected upwards, thereby applying downward force against the flow cell in order to provide efficient thermal transfer from the respective flow cell process heater 106, 108.

The upper face of the flow cell 10 also comes into contact with the retention clip 210 upon insertion. In particular, the downward projection 212 (FIG. 2) of the retention clip rests upon the lid 50 of the flow cell above the third portion 16 and applies downward force on the flow cell, thus bringing the lower face of the flow cell into contact with the heat distribution plate 204 above the heater module 202.

In use, the flow cell 10 is inserted with respect to the flow cell process heater 100 and the flow control heater 200. The flow cell process heaters 106, 108 are brought to temperature. The target DNA and associated reagents are then introduced into the fluid flow path or channel 26, followed by a volume of inert fluid for advancing the sample to the desired position in the fluid flow path or channel with respect to the first and second regions 14, 12 and the respective flow cell process heaters 108, 106. In this embodiment, the retention clip 210 and loading port 20 are configured to not physically interfere upon insertion of the flow cell.

In an alternative embodiment, the target DNA and reagents are inserted into the fluid flow path or channel 26 prior to insertion of the flow cell 10 into the flow cell process heater 100 and the flow control heater 200. In this embodiment, it may be preferable for the flow cell process heaters 108, 106 to already be at a respective desired temperature.

Once the flow cell 10 is installed within the flow cell process heater 100 and with respect to the flow control heater 200, the target DNA and associated reagents have been loaded into the flow cell, and a sufficient quantity of inert fluid has been inserted into the fluid flow path or channel 26 to advance the sample into position with respect to the first and second regions 14, 12, the heater module 202 of the flow control heater is selectively heated or cooled to increase or decrease, respectively, the pressure within the third or middle transport region 16. As pressure is lowered as a result of a lower temperature of the heater module, the sample is moved into the second or denaturation region 12 and maintained at the appropriate temperature for a predetermined period of time. Then, at the appropriate time, the heater module temperature is raised, thus increasing the flow path or channel pressure within the middle transport region, forcing the sample into the first or annealing region 14, where it is maintained for a desire time period. This process is then repeated for a predetermined number of iterations or cycles according to the requirements of the technique being practiced.

This process requires that there be a predetermined relationship between some or all of the temperature of the heater module, the temperature of the flow cell process heaters 106, 108, the volume and configuration of the fluid flow path or channel 26, the viscosity and volume of the sample, and the viscosity and volume of the inert fluid in order for the sample to be properly positioned through the variation in middle transport region pressure. A controller (not shown) in communication with at least the heater module 202 responds according to this predetermined relationship for causing the heater module to output the necessary heating. Feedback may be provided by the temperature sensor 220.

Many different arrangements of the various components depicted, as well as components not shown, are possible without departing from the spirit and scope of the disclosed technology. Embodiments of the disclosed technology have been described with the intent to be illustrative rather than restrictive. Alternative embodiments will become apparent to those skilled in the art that do not depart from its scope. A skilled artisan may develop alternative means of implementing the aforementioned improvements without departing from the scope of the disclosed technology.

For example, while two flow cell process heaters 106, 108, and two complimentary heater portions 12, 14 on the flow cell, are described and illustrated, more than two heaters and a like number of heater portions may be employed, depending upon the application. The sample may be positioned with respect to such an arrangement in the same manner, through the selective application of heating and cooling at the third heater portion 16 of the flow cell via the flow control heater 200.

In addition, a single, continuous fluid flow path or channel 26 on the flow cell 10 has been disclosed and described. However, in an alternative embodiment, plural such fluid flow paths or channels could be arranged interleaved and in parallel, whereby respective samples are processed in the context of the same flow cell process heaters, or can each be disposed on a respective, discrete portion of the flow cell. The latter embodiment would then require plural consecutively arranged flow cell process heater modules and a like number of flow cell heaters.

It will be understood that certain features and sub-combinations are of utility and may be employed without reference to other features and sub combinations and are contemplated within the scope of the claims. Not all steps listed in the various figures need be carried out in the specific order described.

I claim:

1. A flow cell for DNA amplification, comprising:
a substrate having a proximal end, a distal end opposite the proximal end, a first side intermediate the proximal end and the distal end, a second side opposite the first side and intermediate the proximal end and the distal end, and opposite upper and lower faces each bound by the proximal end, the distal end, the first side, and the second side; and
a continuous fluid flow channel disposed on the upper surface, the fluid flow channel comprising
a loading port,
a first heat portion in fluidic communication with the loading port, proximate the distal end and the first side, and configured for being heated to a first temperature,
a second heat portion in fluidic communication with the first heat portion, proximate the distal end and the second side, and configured for being heated to a second temperature, and
a third heat portion in fluidic communication with the second heat portion, intermediate the first side and the second side, and configured for being heated to one of a plurality of temperatures.

2. The flow cell of claim 1, wherein the substrate is rectangular.

3. The flow cell of claim 1, wherein the fluid flow channel further comprises a fourth unheated portion in communication with the first heat portion and proximate the proximal end of the substrate.

4. The flow cell of claim 1, further comprising a first thermal barrier intermediate the first heat portion and the third heat portion and intermediate the second heat portion and the third heat portion.

5. The flow cell of claim 4, wherein the first thermal barrier comprises a first barrier portion intermediate the first heat portion and the third heat portion and a second barrier portion intermediate the second heat portion and the third heat portion.

6. The flow cell of claim 4, wherein the first thermal barrier is disposed on the substrate.

7. The flow cell of claim 1, further comprising a second thermal barrier intermediate the first heat portion and the second heat portion.

8. The flow cell of claim 7, wherein the second thermal barrier is a discontinuity in the distal end of the substrate.

9. The flow cell of claim 1, further comprising a lid over the upper face, the loading port forming an orifice through the lid and into the fluid flow channel.

10. The flow cell of claim 1, wherein the second temperature is greater than the first temperature.

11. The flow cell of claim 1, wherein the loading port is intermediate the proximal end and third heat portion.

12. The flow cell of claim 1, wherein the fluid flow channel in the third heat portion is substantially serpentine.

13. The flow cell of claim 1, wherein the fluid flow channel in each of the first and second heat portions is substantially serpentine.

14. A system for DNA amplification, comprising:
a flow cell comprising
a rectangular substrate having a proximal end, a distal end opposite the proximal end, a first side intermediate the proximal end and the distal end, a second side opposite the first side and in the proximal end and the distal end, and opposite upper and lower faces each bound by the proximal end, the distal end, the first side, and the second side,
a continuous fluid flow channel disposed on the upper face, the fluid flow channel comprising
a loading port,
a first heat portion in fluidic communication with the loading port, proximate the distal end and the first side, and configured for being heated to a first temperature,
a second heat portion in fluidic communication with the first heat portion, proximate the distal end and the second side, and configured for being heated to a second temperature,
a third heat portion in fluidic communication with the second heat portion, intermediate the first side and the second side, and configured for being heated to one of a plurality of temperatures, and
a fourth unheated portion in communication with the first heat portion and proximate the proximal end of the substrate;
a flow cell process heater for selectively receiving the distal end of the flow cell, for heating the first heat portion to the first temperature, and for heating the second heat portion to the second temperature; and
a flow control heater proximate the flow cell process heater and configured to selectively heat the third heat portion of the fluid flow channel via the lower face of the flow cell when the distal end of the flow cell is installed within the flow cell process heater,
whereby the selective heating of the third portion of the fluid flow channel causes the pressure in the third portion to increase or decrease relative to the pressure in the fourth unheated portion, based upon the degree of selective heating by the flow control heater, thereby selectively moving fluid within the fluid flow channel between the first heat portion and the second heat portion.

15. The system of claim 14, wherein the flow cell process heater further comprises optical detector windows, whereby the first heat portion and the second heat portion are each visible through a respective one of the optical detector windows when the distal end of the flow cell is received within the flow cell process heater.

16. The system of claim 14, wherein the flow cell process heater comprises a first heater for heating the first heat portion to the first temperature and a second heater for heating the second heat portion to the second temperature.

17. The system of claim 16, wherein each of the first heater and the second heater is an individually controlled Peltier heater.

18. The system of claim 14, wherein the flow cell process heater comprises resilient members for selectively engaging the flow cell when the flow cell distal end is inserted into the flow cell process heater.

19. The system of claim 18, wherein each of the resilient members extends laterally across a top of the flow cell process heater.

20. The system of claim 18, wherein each resilient member is attached to the flow cell process heater at a respective distal end thereof and has a proximal end that is deflected upwards upon insertion of the flow cell into the flow cell process heater, the deflection causing each of the resilient members to apply downward force on the flow cell.

21. The system of claim 14, wherein the flow control heater comprises a heater module and a heat distribution plate intermediate the heater module and the lower face of the flow cell when the distal end of the flow cell is received within the flow cell process heater.

22. The system of claim 21, wherein the heater module is a Peltier module.

23. The system of claim 14, further comprising a resilient flow cell retention clip proximate the flow control heater and deformable upwardly by the upper face of the flow cell when the flow cell is received within the flow cell process heater, the deformation of the resilient flow cell retention clip applying downward force on the flow cell.

24. The system of claim 23, wherein the resilient flow cell retention clip is adjacent the third heat portion of the continuous fluid flow channel when the flow cell is received within the flow cell process heater.

25. The system of claim 23, wherein the flow cell further comprises a lid over the upper face, the loading port forming an orifice through the lid and into the fluid flow channel, the resilient flow cell retention clip is adjacent the lid over the third heat portion of the continuous fluid flow channel when the flow cell is received within the flow cell process heater.

26. The system of claim 14, wherein the second temperature is greater than the first temperature.

27. A method for implementing oscillating flow PCR, comprising:
inserting a flow cell into a flow cell process heater and over a flow control heater,
the flow cell comprising
a rectangular substrate having a proximal end, a distal end opposite the proximal end, a first side intermediate the proximal end and the distal end, a second side opposite the first side and intermediate the proximal end and the distal end, and opposite upper and lower faces each bound by the proximal end, the distal end, the first side, and the second side,
a continuous fluid flow channel disposed on the upper surface, the fluid flow channel comprising
a loading port in communication with the fluid flow channel,
a first heat portion in fluidic communication with the loading port, proximate the distal end and the first side, and configured for being heated to a first temperature,
a second heat portion in fluidic communication with the first heat portion, proximate the distal end and the second side, and configured for being heated to a second temperature,
a third heat portion in fluidic communication with the second heat portion, intermediate the first side and the second side, and configured for being heated to one of a plurality of temperatures, and
a fourth unheated portion in communication with the first heat portion and proximate the proximal end of the substrate,
the flow cell process heater selectively receiving the distal end of the flow cell;
selectively heating the first heat portion of the fluid flow channel to the first temperature via a first heater in the flow cell process heater;
selectively heating the second heat portion of the fluid flow channel to the second temperature via a second heater in the flow cell process heater, the second temperature being greater than the first temperature;
disposing a DNA template and associated reagents into the loading port; and
selectively heating the lower face of the flow cell and the third portion of the fluid flow channel via the flow control heater, thereby changing the pressure within the third portion relative to the pressure within the remainder of the fluid flow channel and moving the DNA template and associated reagents relative to the first heat portion and the second heat portion.

28. The method of claim 27, further comprising disposing a quantity of inert fluid into the loading port after disposing the DNA template and associated reagents into the loading port, the quantity of inert fluid for positioning the DNA template and associated reagents in the fluid flow channel relative to the first heat portion and the second heat portion.

29. The method of claim 28, wherein the inert fluid is one or both of air and mineral oil.

30. The method of claim 27, further comprising sealing the loading port following the step of disposing the DNA template and associated reagents into the loading port.

31. The method of claim 27, where the step of selectively heating the lower face of the flow cell and the third portion of the fluid flow channel via the flow control heater comprises lowering the temperature of the third portion of the fluid flow channel via the flow control heater to a temperature sufficient to lower the pressure within the third portion of the fluid flow channel relative to the pressure in the fourth unheated portion of the fluid flow channel thereby moving the DNA template and associated reagents into the second heat portion, then raising the temperature of the third portion of the fluid flow channel via the flow control heater to a temperature sufficient to raise the pressure within the third portion of the fluid flow channel relative to the pressure in the fourth unheated portion of the fluid flow channel thereby moving the DNA template and associated reagents into the first heat portion.

32. The method of claim 31, each repetition of the steps of lowering and raising the temperature of the third portion of the fluid flow channel defining a cycle, the method further comprising consecutively repeating the cycle a predefined number of times.

33. The method of claim 31, wherein the steps of lowering and raising the temperature of the third portion of the fluid flow channel are each performed for a predetermined period of time.

34. The method of claim 27, wherein the step of inserting a flow cell into a flow cell process heater and over a flow control heater comprises inserting the distal end of the flow cell under a respective first end of resilient members, each resilient member attached at an opposite second end to the flow cell process heater, the first end of each resilient member being deflected upward by the flow cell upon insertion of the flow cell into the flow cell process heater and thereby applying downward force on the flow cell.

35. The method of claim 27, wherein the step of inserting a flow cell into a flow cell process heater and over a flow control heater comprises inserting the flow cell under a resilient flow cell retention clip prior to inserting the distal end of the flow cell into the flow cell process heater, the resilient flow cell retention clip pressing against the third heat portion of the flow cell when the flow cell is inserted into the flow cell process heater, thereby bringing the lower face of the flow cell into thermal contact with the flow control heater.
